(19) United States
(12) Patent Application Publication
(10) Pub. No.: US 2022/0261664 A1
(43) Pub. Date: Aug. 18, 2022

(54) INCREMENTAL RULE CONDITION EVALUATION

(71) Applicant: OptumSoft, Inc., Menlo Park, CA (US)

(72) Inventor: David R. Cheriton, Vero Beach, FL (US)

(21) Appl. No.: 17/591,032

(22) Filed: Feb. 2, 2022

Related U.S. Application Data
(60) Provisional application No. 63/147,130, filed on Feb. 8, 2021.

(51) Int. Cl. G06N 5/02; G06Q 40/06

(52) U.S. Cl. CPC G06N 5/025; G06Q 40/06

(57) ABSTRACT

A first ruleset associated with a rule-based system is received. Incremental rule evaluation is performed, wherein not all input values are known at the start of rule evaluation, at least in part by: selecting a first input for which to determine a first new value; determining the first new value; and determining a second ruleset by removing from the first ruleset rules whose rule condition can no longer be true based on the first input value.

### CROSS REFERENCE TO OTHER APPLICATIONS

This application claims priority to U.S. Provisional Patent Application No. 63/147,130 entitled INCREMENTAL RULE CONDITION EVALUATION filed Feb. 8, 2021 which is incorporated herein by reference for all purposes.

### BACKGROUND OF THE INVENTION

With conventional rule-based systems, all the inputs are assumed available at the point the rule conditions are evaluated, the rule conditions being evaluated to determine the rules that should “fire” at this time. It may be expensive to have all inputs available at the point the rule conditions are evaluated.

### BRIEF DESCRIPTION OF THE DRAWINGS

Various embodiments of the invention are disclosed in the following detailed description and the accompanying drawings.

FIG. 1 is a functional diagram illustrating a programmed computer/server system for incremental rule condition evaluation in accordance with some embodiments.

FIG. 2A is a block diagram illustrating traditional rule evaluation, where all values are available at the start of evaluation.

FIG. 2B is an illustration of an example of a fault scenario vector of symptoms for the RCI application.

FIG. 2C is an illustration of a rule condition table for the RCI application, as a root cause table.

FIG. 3 is a flow diagram illustrating an embodiment of a process of incremental rule condition evaluation.

FIG. 4 is a flow diagram illustrating an embodiment of a process incremental rule condition evaluation when some input values are previously known.

FIG. 5 is an illustration of an embodiment of a conjunctive rule condition table representation.

FIG. 6 is a flow diagram illustrating an embodiment of a process for incremental evaluation of a ruleset.

FIG. 7 is a flow diagram illustrating an embodiment of a process for performing incremental rule evaluation.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Incremental rule condition evaluation is disclosed. As referred to herein, ‘incremental rule condition evaluation’ comprises reducing a ruleset based at least in part by determining a value of an input to one or more rule conditions at evaluation time.

In one embodiment, not all rule inputs are known initially. Reducing the ruleset may minimize the cost of testing/telemetry collection and/or each incremental evaluation significantly reduces the collection of rules that may be expected to fire or be acted upon. In one embodiment, an initial hypothesis (and subsequent hypotheses) of the appropriate subset of the rule actions is accommodated.

In one embodiment, a ruleset is incrementally evaluated by, at evaluation time, selecting an input Ii to one or more rule conditions, determining the value of that input Ii and reducing the ruleset to a subset of this ruleset containing rules that may be true based on the now determined value of input Ii. An illustrative example without limitation is differential diagnosis in medicine and/or medical decision making, to determine one or more patient issues through the process of tests.

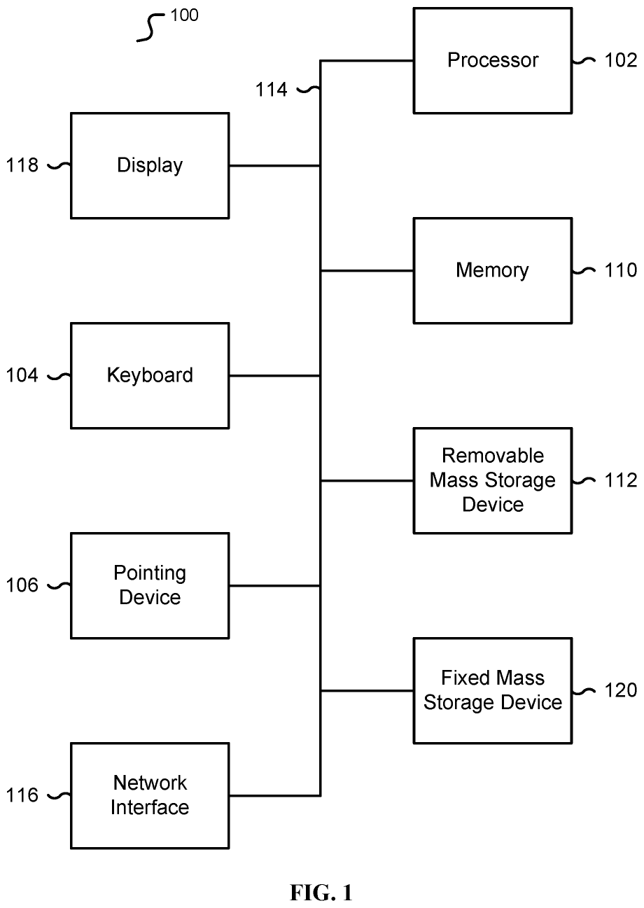

FIG. 1 is a functional diagram illustrating a programmed computer/server system for incremental rule condition evaluation in accordance with some embodiments. As shown, FIG. 1 provides a functional diagram of a general purpose computer system programmed to provide incremental rule condition evaluation in accordance with some embodiments. As will be apparent, other computer system architectures and configurations can be used for incremental rule condition evaluation.

Computer system 100, which includes various subsystems as described below, includes at least one microprocessor subsystem, also referred to as a processor or a central processing unit (“CPU”) 102. For example, processor 102 can be implemented by a single-chip processor or by multiple cores and/or processors. In some embodiments, processor 102 is a general purpose digital processor that controls the operation of the computer system 100. Using instructions retrieved from memory 110, the processor 102 controls the reception and manipulation of input data, and the output and display of data on output devices, for example display and graphics processing unit (GPU) 118.

Processor 102 is coupled bi-directionally with memory 110, which can include a first primary storage, typically a random-access memory (“RAM”), and a second primary storage area, typically a read-only memory (“ROM”). As is well known in the art, primary storage can be used as a general storage area and as scratch-pad memory, and can also be used to store input data and processed data. Primary storage can also store programming instructions and data, in the form of data objects and text objects, in addition to other data and instructions for processes operating on processor 102. Also as well known in the art, primary storage typically includes basic operating instructions, program code, data, and objects used by the processor 102 to perform its functions, for example, programmed instructions. For example, primary storage devices 110 can include any suitable computer-readable storage media, described below, depending on whether, for example, data access needs to be bi-directional or uni-directional. For example, processor 102 can also directly and very rapidly retrieve and store frequently needed data in a cache memory, not shown. The processor 102 may also include a coprocessor (not shown) as a supplemental processing component to aid the processor and/or memory 110.

A removable mass storage device 112 provides additional data storage capacity for the computer system 100, and is coupled either bi-directionally (read/write) or uni-directionally (read-only) to processor 102. For example, storage 112 can also include computer-readable media such as flash memory, portable mass storage devices, holographic storage devices, magnetic devices, magneto-optical devices, optical devices, and other storage devices. A fixed mass storage 120 can also, for example, provide additional data storage capacity. One example of mass storage 120 is an eMMC or microSD device. In one embodiment, mass storage 120 is a solid-state drive connected by a bus 114. Mass storages 112, 120 generally store additional programming instructions, data, and the like that typically are not in active use by the processor 102. It will be appreciated that the information retained within mass storages 112, 120 can be incorporated, if needed, in standard fashion as part of primary storage 110, for example RAM, as virtual memory.

In addition to providing processor 102 access to storage subsystems, bus 114 can be used to provide access to other subsystems and devices as well. As shown, these can include a display monitor 118, a communication interface 116, a touch (or physical) keyboard 104, and one or more auxiliary input/output devices 106 including an audio interface, a sound card, microphone, audio port, audio input device, audio card, speakers, a touch (or pointing) device, and/or other subsystems as needed. Besides a touch screen, the auxiliary device 106 can be a mouse, stylus, track ball, or tablet, and is useful for interacting with a graphical user interface.

The communication interface 116 allows processor 102 to be coupled to another computer, computer network, or telecommunications network using a network connection as shown. For example, through the communication interface 116, the processor 102 can receive information, for example data objects or program instructions, from another network, or output information to another network in the course of performing method/process steps. Information, often represented as a sequence of instructions to be executed on a processor, can be received from and outputted to another network. An interface card or similar device and appropriate software implemented by, for example executed/performed on, processor 102 can be used to connect the computer system 100 to an external network and transfer data according to standard protocols. For example, various process embodiments disclosed herein can be executed on processor 102, or can be performed across a network such as the Internet, intranet networks, or local area networks, in conjunction with a remote processor that shares a portion of the processing. Throughout this specification, “network” refers to any interconnection between computer components including the Internet, Bluetooth, WiFi, 3G, 4G, 4GLTE, GSM, Ethernet, intranet, local-area network (“LAN”), home-area network (“HAN”), serial connection, parallel connection, wide-area network (“WAN”), Fibre Channel, PCI/PCI-X, AGP, VLbus, PCI Express, Expresscard, Infiniband, ACCESS.bus, Wireless LAN, HomePNA, Optical Fibre, G.hn, infrared network, satellite network, microwave network, cellular network, virtual private network (“VPN”), Universal Serial Bus (“USB”), FireWire, Serial ATA, 1-Wire, UNI/O, or any form of connecting homogenous and/or heterogeneous systems and/or groups of systems together. Additional mass storage devices, not shown, can also be connected to processor 102 through communication interface 116.

An auxiliary I/O device interface, not shown, can be used in conjunction with computer system 100. The auxiliary I/O device interface can include general and customized interfaces that allow the processor 102 to send and, more typically, receive data from other devices such as microphones, touch-sensitive displays, transducer card readers, tape readers, voice or handwriting recognizers, biometrics readers, cameras, portable mass storage devices, and other computers.

In addition, various embodiments disclosed herein further relate to computer storage products with a computer readable medium that includes program code for performing various computer-implemented operations. The computer-readable medium is any data storage device that can store data which can thereafter be read by a computer system. Examples of computer-readable media include, but are not limited to, all the media mentioned above: flash media such as NAND flash, eMMC, SD, compact flash; magnetic media such as hard disks, floppy disks, and magnetic tape; optical media such as CD-ROM disks; magneto-optical media such as optical disks; and specially configured hardware devices such as application-specific integrated circuits (“ASIC”s), programmable logic devices (“PLD”s), and ROM and RAM devices. Examples of program code include both machine code, as produced, for example, by a compiler, or files containing higher level code, for example a script, that can be executed using an interpreter.

The computer/server system shown in FIG. 1 is but an example of a computer system suitable for use with the various embodiments disclosed herein. Other computer systems suitable for such use can include additional or fewer subsystems. In addition, bus 114 is illustrative of any interconnection scheme serving to link the subsystems. Other computer architectures having different configurations of subsystems can also be utilized.

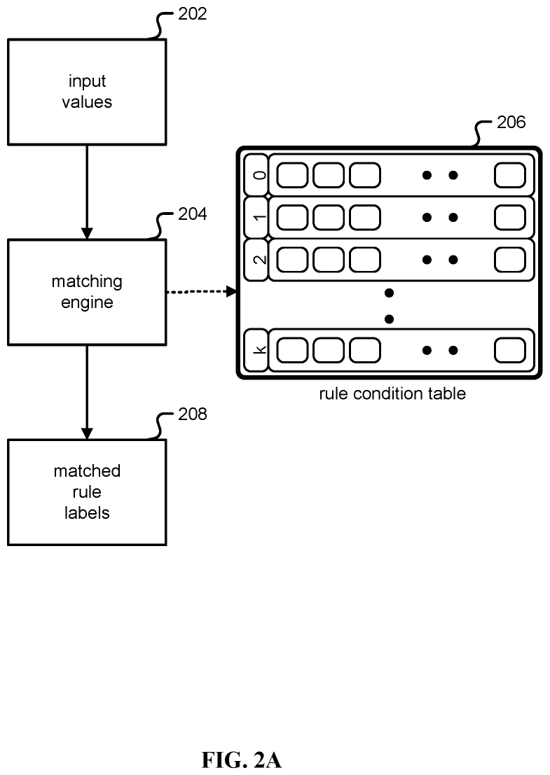

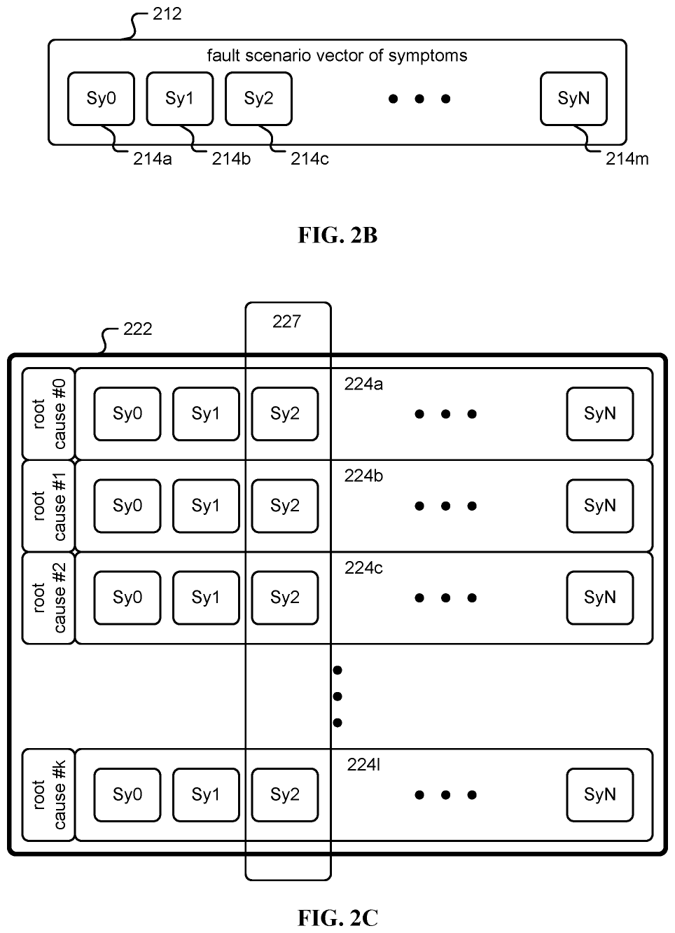

FIG. 2A is a block diagram illustrating traditional rule evaluation, where all values are available at the start of evaluation. In traditional rule-based systems, all inputs are assumed available at the point the rule conditions are evaluated. Rule conditions may be evaluated to determine the rules that should “fire” at evaluation time. For instance, root cause analysis or root cause identification (RCI) may be performed by a collection of rules that each match a set of conditions or symptoms and output a specific root cause fault when there is a match, that is the rule condition evaluates to true.

Root Cause Analysis. Actual root causes associated with a given actual fault scenario in FIG. 2A as an input value (202) are determined by using a matching engine (204) to match the given actual fault scenario against each row in the rule condition table such as a root cause table (206), and indicating the rows that match, in the root cause application as probable root causes. That is, if an input value/actual fault scenario matches a row such that each entry matches via a match (a,b) operator described below, the root cause associated with that row is output as a matched rule label (208) associated with this fault scenario/input value, as shown in FIG. 2A.

FIG. 2B is an illustration of an example of a fault scenario vector of symptoms for the RCI application. One example of a symptom, noPower, is a symptom indicating that there is no power coming to a monitored system. The state of a symptom may be a known value or a special indication that it is unknown and/or “don't care.” The term “don't care” is commonly used in digital logic to indicate that the associated item is extraneous/not required. The ability for the processing to indicate “don't care” for a given symptom allows analysis to proceed even when that aspect of the state of the system is not actually known.

A “fault scenario” is referred to herein as a collection of symptom values that indicates the known and unknown fault state of a monitored system. Logically a fault scenario represents the state and/or potential partial state of the system from the standpoint of observed/determined symptoms that something is wrong or not wrong with the system. It may not indicate the full state of the system. For example, with a vehicle, the fault scenario may not necessarily indicate the position, velocity, and so forth of the vehicle, but may only the state of the symptoms, that is, the aspects that are needed to perform root cause analysis of faults.

As shown in FIG. 2B, in one embodiment, a fault scenario is represented as an array of values (212), where each entry (214a-m) corresponds to a specified symptom. For example, symptom Sy0 (214a) is a first entry, symptom Sy1 (214b) is a second entry, and so forth. In one embodiment, there may be multiple symptoms associated with the same metric. For example, there may be different symptoms for a temperature sensor being slightly high, moderately high, and extremely high. In one embodiment, there may be symptoms associated with the same metric based on different levels of derivative. For example, a symptom may be associated with a metric having a first derivative that is zero for too long, that is, it is constant, often indicating that the input sensor has failed. A symptom may be associated with the first derivative being too high, meaning that it is changing too quickly.

There may be additional symptoms associated with a metric that indicate that the metric is out-of-range or behaving incorrectly. In this case, the out-of-range symptom is set at the same time as a symptom indicating the metric is too high or too low, for instance. This “aggregate” form of symptom may allow a fault scenario to be specified in terms of “out of range,” rather than having to cover both “too low” and “too high.”

A match operator is defined herein between two fault scenarios s0 and s1 to return true

bool isMatching=match(s0,s1);

if every symptom entry in s0 is either “don't care” or else matches as the value in the corresponding entry in s1. Note that the match operation is not commutative; match (a,b) may not necessarily be equal to match (b,a).

FIG. 2C is an illustration of a rule condition table for the RCI application, as a root cause table (RCT). An RCT is a table in which each row is a fault scenario that is labeled with an associated root cause. In this context, an unknown value for a symptom in such a fault scenario is interpreted as “don't care.” For example, for a root cause “front obstacle,” symptoms in the row may be: vehicleInFront as true, movingForward as true, and all other symptoms indicated as “don't care.”

In one embodiment, an RCT contains a row for every failure or event that can be the root cause, where each row indicates the symptoms that must be true for this to be the root cause, those that must be false, and the rest set as indicating “don't care.” Note that specifying more symptoms as specific values, rather than “don't care” beyond the absolute minimal for a given root cause can result in a root cause not being identified or matched because extra symptoms may not be known or are the opposite of that specified for the row. Consequently, it is important to specify the minimal set of known symptoms required to diagnose the system to the particular root cause associated with the row in the table. If a given root cause may have multiple identifying sets of symptoms, there are multiple rows in the RCT, as a row per set. A given root cause may have multiple corresponding rows because one row corresponds to a minimal set of symptoms and others correspond to the minimal set with additional symptoms that provide greater confidence in the root cause. For example, in the case of a power supply failure to a switch, the minimal set may just contain the “lossOfPower” symptom from the switch's current sensor while additional rows may contain that symptom plus “lossOfSignal” symptoms from the directly attached switches to the failed switch.

In one embodiment, each RCT row is represented in the same way as a fault scenario. As such, it may be referred to herein as a “potential fault scenario.” As shown in FIG. 2C, an RCT (222) comprises k+1 rows (224a-5241), each row associated with a specific root cause with N symptoms per row. For example, root cause #0 is associated with the first row (224a). The values of the symptoms (504a-m) in each row (224a) are different from the other rows (224b-5241), each corresponding to a potential fault scenario for the associated root cause, as indicated by the root cause labeled #0 through #k.

In contrast to a potential fault scenario, the fault scenario determined from a monitored system is referred to herein as an “actual fault scenario”. There may be multiple actual fault scenarios for a monitored system. One actual fault scenario may be a more detailed fault scenario for a particular subsystem compared to another. Another source of multiple actual fault scenarios is uncertainty regarding the faults. For example, one scenario may have a symptom corresponding to the temperature of the system being too low whereas another may have a symptom indicating that the temperature sensor has failed. In the latter case, it may indicate the temperature sensor-dependent symptoms as unknown.

As described herein, ternary symptom values are used so that a symptom is represented as a “known” bit indicating known or unknown by being true or false respectively, and a second “value” bit that indicates true or false, which is only interpreted as such if the known bit is set to true. The quaternary nomenclature of [a, b] is used, again such that an interpretation of [0,1] that is allowable is that an associated symptom is not known to be true. Thus [0,0] which may correspond to unknown differs from [0,1] which may be interpreted as not known to be true. Note that a [0,1] symptom in an entry in an RCT (222) may match to an input being false or unknown unlike [0,0], which just does not match to true. Again, [0,1] may not necessarily be treated the same as [0,0] and/or not allowed.

Binary, ternary, and quaternary representation. In one embodiment, a value has three or more states, including the values of “true,” “false,” and “don't care.” In one embodiment, ternary subcondition values are used so that a subcondition is represented as a “known” bit indicating known or unknown by the bit value being true or false respectively, and a second “value” bit that indicates true or false, which is only interpreted as such if the “known” bit is set to true. A quaternary representation using two bits to represent a ternary value is referred to herein as [a, b] wherein a is whether a state is known (0=unknown, 1=known) and b is a value associated with the state (0=false, 1=true). With this convention, an interpretation of [0,1] that is allowable is that an associated subcondition is “not known to be true.” Compare [0,0], which may correspond to the state of unknown, with [0,1], which may be interpreted as “not known to be true” (An alternate convention may be that [0,0] is “not known to be false”.) Completing the possibilities, a [1,0] may correspond to be “false” and [1,1] may correspond to be “true” . . . ” Note that a [0,1] subcondition may match to an input that is false or unknown, and is unlike [0,0], which does not match any input to true. Thus [0,1] may not necessarily be treated the same as [0,0] and/or may not be allowed in an internal and/or consistent representation.

In one embodiment, a value has two states that allow for different matching behavior for different columns of the table. For a value with binary states, in many applications an object has a symptom or else the clients “don't care,” for example an object representing isOverheating (that is, whether a unit is overheating). A “true” for isOverheating indicates a symptom for a particular issue, but there may be many root causes that are not related to whether the unit overheats and is thus “don't Care”. Recognizing this, an alternative approach is to have two values per entry rather than three, corresponding to “true” and “don't care.” A corresponding match operator output table is:

Table Entry

| Input | “don't care” | “true” |
|---|---|---|
| “don't care” | match | no-match |
| “true” | match | match |

This reduces the number of bits required per entry from two bits for a ternary representation to one bit for binary representation, as is thus half as expensive in memory.

There are cases in which there is a need to match on both an entity having a symptom S1 as well in a separate rule/root cause, not having a symptom, such as S1 is false. For this case, an extra symptom S2 may be explicitly introduced that corresponds to the negation of S1, for example a symptom S1 may be lossOfPower and S2 may be hasPower. Then, a row that requires lossOfPower may have the corresponding table entry for symptom S1 set to “true” and S2 set to “don't care.” Conversely, a row that requires hasPower may have that corresponding table entry for S2 set to “true” and the S1 entry set to “don't care.” Thus, if for example ten percent of the symptoms require negation, a 1-bit approach would still be less expensive in space than the 2-bit approach, that is, an extra 10 percent space but not 100 percent more.

In one embodiment, a combination of S1 and S2 both being true may correspond to “unknown.” That is, if the input sets both S1 and S2 then the symptom is treated as “unknown.” Representing a state as “unknown” may be more useful in applications than a “not known to be true” interpretation of a fourth value. There is no change or extension to the above matching for this “unknown” support to work. The S1 and S2 entries in a row both specify true, and the input specifies these entries as true as well, so it matches on “unknown.”

There are also symptoms that are effectively a discrete enumeration of values, such as very cold, cold, cool, normal, warm, hot, and very hot. These seven values may be represented in three bits in a normal binary representation. However, with the above “true”/“don't care” binary approach, one would need six symptoms for the positive and the negation as separate symptoms for each bit. In particular, the above 7 enumerations can be represented in 3-bits using a normal Boolean representation, e.g. 001 could designate “very cold”. However, because the true/don't care embodiment does not explicitly handle false, another 3 bits or symptoms are required are required to specify false, that is 0. Therefore, the 001 encoding for “very cold” would be indicated the six symptoms [X,X,1,1,1,X] here “X” designates “don't care” and the first 3 entries correspond to true or 1 values and the second 3 entries correspond to the false or 0 entries. Alternately, seven separate symptoms for a “one-hot” representation of these seven values may be used, where the “one-hot” representation is referred to herein as a group of bits wherein legal combinations of values are only those with a single “true” bit and all the others not “true”.

In one embodiment, a specific set of columns used by symptoms representing such an enumeration of multiple symptom values is used. In this case, specific columns are designated as being matched as “true”/“false,” as opposed to “true”/“don't care” to represent the enumeration in three columns, rather than six or seven. There is still a need for a “don't care” value so that a row that does not care about the value of this enumeration symptom may indicate this. Thus, the designation of a column value width is allowed and one value reserved to indicate “don't care,” for example, all zeros. In this case, a logical column width of three bits may be designated so [0,0,0] may correspond to “don't care”, while the remaining seven combinations of values represent the seven different symptom states respectively.” Therefore, this enumeration may be represented in three bits, yet still allows “don't care” to be specified in rows that do not care about the enumeration symptom.

Therefore, this additional extension allows the table designation of logical column width and treating the all zeros case as “don't care.” Then, the columns corresponding to isOverheating or S1, S2 may be tagged as one-bit width whereas the columns corresponding to the above enumeration would be tagged as one logical column of three bits.

In one embodiment, an efficient representation of column width is having a bit per column that is set if the next column is also part of a logical column/entry spanning multiple columns. Equivalently, the bit may indicate a continuation of the previous column. Thus, the columns corresponding to the enumeration may be tagged as [1,1,0], wherein the last bit indicates that there is no continuation beyond the third column to indicate three-bit columns that form one logical column/entry.

Generally, the table may designate column widths as well as the matching behavior for a logical column. As an example of this more general case, the table may designate a logical column as being five bits wide and the matching behavior being a test for “greater than.” That is, the input may match on this entry if its value in these five bits was a binary value that was greater than the value in the table entry, except treating the table entry as “don't care” if it is zero or as “unknown” if it is all ones. As another example of specified matching behavior, it may designate the matching behavior as the logical OR of the input across these columns. By having one bit in the logical column that indicates whether the symptom is relevant to this row and excluding that from the OR, the all zeros value in a table entry may be treated as “don't care.”

In one embodiment, hardware instructions such as the “ANDN” or logical AND NOT instruction in the Intel and AMD instruction set may be used with a binary representation. Hardware instructions are generally faster to execute and more efficient to program than their software counterparts. The ANDN instruction performs a bitwise logical AND with an inverted first operand and a second operand, for example, with the result of mismatch for two operands

inBlock and tableBlock as follows:
mismatch=˜inBlock & tableBlock
   with corresponding output table:

tableBlock

| inBlock | “don't care” = 0 | “true” = 1 |
|---|---|---|
| “don't care” = 0 | mismatch = 0 | mismatch = 1 |
| “true” = 1 | mismatch = 0 | mismatch = 0 |

and thus if mismatch is the complement of match,

tableBlock

| inBlock | “don't care” = 0 | “true” = 1 |
|---|---|---|
| “don't care” = 0 | match | mismatch |
| “true” = 1 | match | match |

This matching may be “ternary matching” but unlike the ternary matching provided by a ternary content-addressable memory (T-CAM), the input fault scenario (202) may also be ternary. A T-CAM may however be used as part of an efficient/hardware system of matching. There may be multiple simultaneous matched rule labels/root cause failures in a monitored system. Therefore, it is possible that the matching matches multiple rows in the table (206), one per matched rule label/root cause. For example, a motor may fail at the same time that a temperature sensor has failed by indicating completely unrealistic readings. There may be multiple rows that map to the same root cause. This handles the case in which a root cause failure may be indicated by different sets of symptoms.

In one embodiment, the row representation may store “don't care” values, where does not explicitly store “don't care” entries. That is, the absence of an explicit designation or representation of an i-th symptom is interpreted as “don't care” for the i-th symptom. In one embodiment, symptoms are aggregated into blocks that are associated with logical units or components of a monitored system. For example, an embodiment may use a 64-bit block of known/value bits. Thus, if a component is not relevant to a particular root cause, the entire block need not be stored. Each row may then require a relatively small amount of storage. Typically, most rows are relatively sparse because only a small subset of symptoms is relevant to a particular fault so only a small percentage of that row is actually stored, with the rest by default being “don't care.”

The representation of arbitrary fault criteria is achieved by using multiple symptoms. For example, one root cause is evidenced by a temperature being very high, yet another is evidenced by it being high, and another evidenced by it being slightly high. That is, there may be a symptom entry in each row for each of these levels.

A key element is indicating the symptoms that are known to be false as a symptom, that is no fault, as well as what is known to be true, that is a fault is present, while still allowing for unknown or “don't care.” The false case effectively filters out symptoms that are due to another reason, for example the compressor is not working, but actually there is no power, which is the root cause. Thus, a subsystem SSi that is dependent on a number of other subsystems may need to have all these other systems known to be working before a fault in subsystem SSi may be reliably identified as a root cause.

In one embodiment, the system may record if any symptoms changed in an actual fault scenario since its last matching and only re-match the actual fault scenario to the RCT (522) if so. This check avoids the overhead of re-matching when there is no change to the actual fault scenario.

In one embodiment, the frequency of re-matching is configurable according to application requirements. For example, ARCA matching may be configured to be performed every 30 minutes in a refrigeration system to minimize the cost of the matching because a fault does not result in immediate problems and the resulting delay in detecting a fault does not significantly impact the meantime to repair. This low rate of matching assumes that a fault that is transient enough to appear and then disappear before the matching takes place, and thus is not present in the actual fault scenario at the time of the matching is not critical to detect.

For example, an experienced network operator may look at the symptoms being provided by telemetry to determine the root cause fault in a computer network. This approach may be computerized to provide automated root cause identification, as described in U.S. Pat. No. 10,761,921, entitled AUTOMATIC ROOT CAUSE ANALYSIS USING TERNARY FAULT SCENARIO REPRESENTATION filed May 8, 2018 which is incorporated herein by reference for all purposes. However, conventional rule evaluation assumes that all the telemetry is available at the time of evaluation, that is at the time of the RCI processing.

In some applications, it is expensive to have all the telemetry available for each evaluation time. For example, in a medical diagnosis, that is medical decision making, some tests are expensive and may introduce significant delay in getting results. For instance, a blood test incurs the cost and delay of laboratory work as well as some discomfort and risk to the patient. A medical procedure may be to determine a hypothesis for a patient issue based on readily available symptoms such as patient appearance, body temperature, heart rate, and/or blood pressure, and then order one or more additional tests to confirm the hypothesis to a diagnosis, with the test choices influenced by the cost and time delay to get the test results. Thus, a blood test, for example, is called for if the patient disorder is not clear from the simple readily available symptoms.

As a further complication, some tests or telemetry provide in their results a different amount of benefit depending on the situation. For example, again with medical diagnosis, different patients may have different levels of benefit from a given test, and there may be different costs and risks to perform a given test for each patient. Therefore, the order of tests to perform may dependent on the specific situation, namely the specific patient in the medical diagnosis example.

So-called expert systems were developed in the 1980's as part of the Stanford Heuristic Programming Project demonstrated success for medical diagnosis (mycin, dendral) as well as analysis of submarine sonar signals and other applications. U.S. Pat. No. 10,761,921 indicates how to do efficient root cause identification. In general, rule-based systems have been shown to be a useful means of analyzing a situation and determining a course of action. However, they traditionally assume that all inputs are available at the time of rule condition evaluation.

Medical Illustration. A simplified example of a ruleset for diagnosing diseases without limitation is as follows:

TABLE A
Full ruleset

| | | | |
|---|---|---|---|
| If | pale && high temperature && low-energy | Then | has flu |
| If | runny nose && stuffy nose && normal | Then | has cold |
| | temperature && cough | | |
| If | high temperature && cough && no runny | Then | has COVID-19 |
| | nose | | |
| If | runny nose && sniffles && normal | Then | has hayfever |
| | temperature | | |
| If | high temperature && high white cell | Then | has blood |
| | count && pale | | infection |

If a (first) test selected to be performed is measurement of the temperature of the patient and the result of this test is the determination that the patient has a high temperature, a subset of this original ruleset is produced. The rules in this subset correspond to those rules whose conditions may be true after the temperature value is known to be “high temperature”, and has been reduced from five rules in the ruleset above, to three rules in the ruleset below. One benefit of this reduction is reducing the number of possible diagnosis and providing clarify on the best test to select next. In this example, determining next whether the patient is pale or not may reduce it to a single diagnosis if the result is negative. Another example may be a patient who through reduction only requires three medical tests rather than all 100 tests contemplated in the original ruleset. This reduction of ruleset is also a technical improvement which allows reduced memory usage, reduces network bandwidth for communicating rulesets, and improves the speed of processing as it reduces the number of rules to be processed by a computer. For example, the amount of processing required to complete the rule evaluation is reduced compared to re-evaluating the entire rule set after incrementally acquiring each additional input. Thus, a step of freeing memory usage associated with the first ruleset may optionally be performed after the incremental rule evaluation.

TABLE B
Reduced ruleset

| | | | |
|---|---|---|---|
| If | pale && high temperature && low-energy | Then | has flu |
| [symbol] | [symbol] | [symbol] | [symbol] |
| | [symbol] | | |
| If | high temperature && cough && no runny | Then | has COVID-19 |
| | nose | | |
| [symbol] | [symbol] | [symbol] | [symbol] |
| If | high temperature && high white cell | Then | has blood |
| | count && pale | | infection |

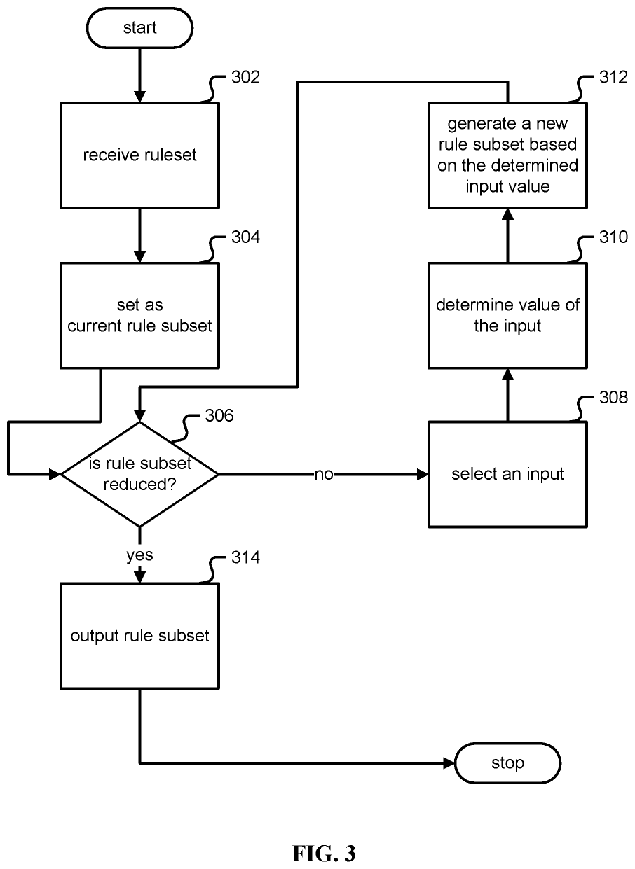

FIG. 3 is a flow diagram illustrating an embodiment of a process of incremental rule condition evaluation. In one embodiment, the process of FIG. 3 is carried out by the system of FIG. 1.

In step (302), a ruleset is received for an application. For the medical example given above, such a ruleset is shown in Table A. In step (304), the ruleset received in step (302) is designated the “current” subset.

In step (306), while the current subset is not sufficiently reduced, the steps (308), (310), and (312) are performed:

In step (308), an input is selected for determination. For the medical example given above, a high temperature test is such a selected input.

In step (310), the input value for the selected input of step (308) is determined. For the medical example given above, the result of the test is that the patient has a high temperature.

In step (312), the new reduced rule subset is determined by removing those rules whose rule condition may no longer be true based on this input value. For the medical example given above, the new reduced rule subset is shown in Table B.

In step (314), the current subset is output. In an optional step not shown in FIG. 3, the system may evaluate and/or trigger the rules in the subset of step (314).

In one embodiment, the reduction of the current subset may be considered sufficient in step (306) when there is no test that produces a smaller subset. In this case, all the rule conditions for the rules in this subset may be true, so the associated rule actions may be performed. For instance, in an RCI system, each rule has an action to report the associated root cause fault. There may be multiple/independent root cause faults at the same time so it is possible that the sufficiently reduced subset may contain multiple rules. This scenario may also arise in medical diagnosis where a patient may have multiple disorders, such as COVID-19 and a blood infection in the above medical example.

In one embodiment, the rule subset may be considered sufficiently reduced in step (306) if all the rules with overlapping undetermined inputs have a common action. For the above medical example, if all the rules remaining in the current subset call for a blood transfusion because that is the primary treatment for the flu, COVID-19, and a blood infection in Table B, the ruleset reduction may be deemed sufficient even though there are additional inputs in the rule conditions whose values have not been determined yet.

In one embodiment, the subset may be considered sufficiently reduced in step (306) when the urgency of some action is more important than the cost or delay with determining additional input values. For example, in a medical domain, if a ruleset includes a potential diagnosis of heart attack, it may be better to start an intervention for heart attack than wait to get the results of additional tests. Similarly, if a piece of industrial mechanical equipment proceeds to make significant frictional noise, it may be better to lubricate the equipment or shut it down immediately before the full diagnosis of the problem has been achieved.

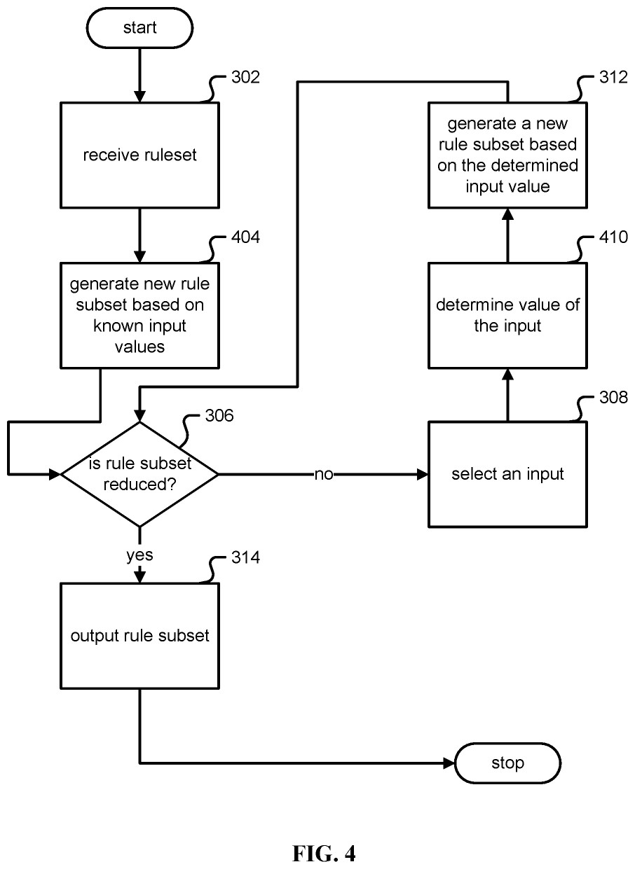

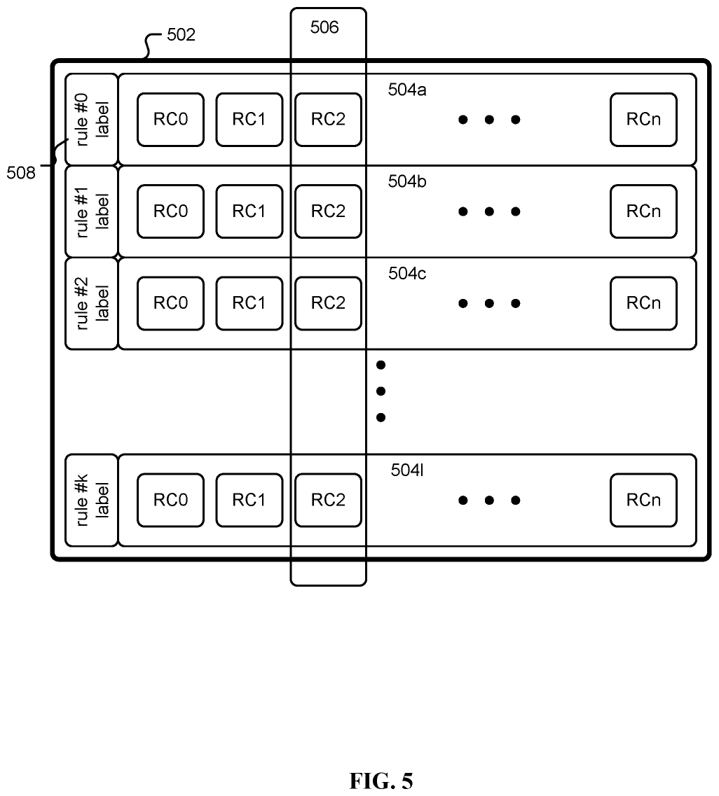

FIG. 4 is a flow diagram illustrating an embodiment of a process incremental rule condition evaluation when some input values are previously known. In one embodiment, the process of FIG. 4 is carried out by the system of FIG. 1. As shown in FIG. 4, the process of FIG. 4 is similar to that of the process of FIG. 3, with similar steps (302), (306), (308), (310), (312), and (314).

In one embodiment, in step (404) some number of input values are known at the start of the rule evaluation. In this case, these input values are used to define the initial current subset by eliminating those rules that cannot possibly be true given the input values, as illustrated in FIG. 4. For example, with medical diagnosis, a patient may have a number of evident or self-reported symptoms such as paleness, high temperature and headache when this person shows up with a complaint. This initial reduction removes all the rules that cannot be true based on these pre-determined input values, producing the initial current subset. In the medical example above, if the patient self-reports a runny nose, then the rule associated with “no runny nose” in Table A for COVID-19 may be eliminated.

Also, a patient may have a known pre-existing condition or a medical history indicating, for instance, high blood pressure. The pre-existing condition may be handled by using this condition to imply certain symptoms/input values as predetermined, and use these predetermined values to reduce the current subset as above. The pre-existing condition is still included in this subset, but may be removed, given it is already known, from the final reduced subset to isolate the new/unknown condition. In the medical example above, there may be a medical history of a patient with a propensity for blood infection, for example, which may imply symptoms/input values like a high temperature.

Input Test Selection. The step (308) of selecting, at iteration of the process in FIGS. 3 and/or 4, an input to test may use the process of various rule-based decision tree generation for attribute selection. Attribute selection may be considered selecting an input to an actual rule, assuming a rule condition is a conjunction of “attri=vj”. For example, RBDT-1 by Abdelhalim, Traore, and Sayed is a traditional Rule-Based Decision Tree generation technique that uses attribute effectiveness, attribute autonomy, and/or minimum value distribution as criteria to select the attribute. These are used as a heuristic to select the attribute at each stage that builds the decision tree with the lowest number of nodes.

In general, traditional techniques focus on achieving the maximum information gain at each stage. As referred to herein, expected information gain IG is the reduction in information entropy H from a prior state to a state that takes some information as given,

IG(T,a)=H(T)−H(T|a)

where H(T|a) is the conditional entropy of T given the value of attribute a.

For example, an attribute with M possible values that reduces the number of possibilities in each subtree by a factor of M provides good information gain whereas picking an attribute that is merely relevant to a single outcome only reduces one side of the decision tree by one leaf node and is not considered good information gain. With good information gain, the size of the resulting/remaining, ruleset is significantly reduced, thereby providing incremental benefit as a result of the incremental evaluation.

In one embodiment, the attribute selection is based at least in part on the cost associated with the selection. For instance, the information gain may be divided by the cost of determining the attribute to factor in the cost. Alternatively, the cost may be subtracted from the information gain. The cost may include the dollar cost to conduct the test to determine the attribute value as well as the cost of delaying the diagnosis in order to perform a test that takes some time. On the other hand, the information gain portion may be extended to include the risk reduction associated with performing the test. For example, in medical diagnosis, if there is a risk of a brain aneurysm in a patient, there is a significant cost to delaying the diagnosis based on the cost of tests.

In one embodiment, the score of an attribute test AT may be without limitation a function of one or more variables, such as:
AT_{score}=f (IG_{AT}),
AT_{score}=g(RR_{AT}),
AT_{score}=h(TC_{AT}), and/or
AT_{score}=m(TD_{AT})
   or a combination of one or more of those variables, for example:

AT_{score}=K₀IG_{AT}K₁RR_{AT}−(K₂TC_{AT}+K₃TD_{AT})

where
K₀, K₁, K₂ and K₃ are constants;
IG_{AT} is the expected information gain from this test;
RR_{AT} is the expected risk reduction from performing the AT test;
TC_{AT} is the cost of the AT test; and
TD_{AT} is the cost of the deferring diagnosis until the results of test AT are available.

In one embodiment, the input attribute to determine in step (308), and thus the test to perform, is based on the input attribute that has the highest score across all the undetermined input attributes.

Note that the score computed is used to compare against the value computed for other inputs, so there need not be meaningful units associated with the computation. Also, it may not be possible to consistently pick the optimum attribute or test at each step because the result of the test is not known in advance. For instance, one test with a particular result may reduce the rule subset to a single rule but if the result is not that particular value, it may provide little information. An objective is therefore to achieve the best results over a number of rule evaluation instances.

The values of the above constants and the other values may be specific to a scenario, and thus dynamically determined at evaluation time. For example, in the medical example above, the cost of a particular test may depend on a particular patient's medical insurance and the variable risk that is dependent on the particular patient's age. In this case, it is not feasible to determine the order of tests in advance of rule evaluation on a specific scenario, or patient in the medical context.

If it is not feasible to perform a given test in a particular scenario or because of some failure or condition in the testing capability, that test may be indicated as very high cost so it is not selected. It is also feasible to support the operator exploring “what-if” scenarios by being able to specify a given test and test result at any point to see what the effect on the rule subset is.

In an application in which a ruleset is required to produce one action or the “first match” of a rule condition is adequate to use, a decision tree may be automatically produced from the ruleset using traditional techniques. An improvement in the more general case of a dynamic environment and/or where attribute selection depends on a particular scenario (or patient in the case of medical diagnosis), the process of FIGS. 3 and/or 4 is more efficient, as traditionally the entire decision tree is generated in advance, yet only the path to the decision is used. It may also not be feasible to reuse the generated decision tree.

By contrast, merely incurring the overhead of selecting the input attributes and thus decision nodes that are used in a given scenario rather than the entire decision tree is disclosed.

In many applications such as root cause analysis and diagnosis, there may be more than one fault or disorder, so the “first-match” semantic is not adequate, and thus the decision tree approach is not viable. Therefore, allowing test sequence construction that is dynamic, individualized, and/or specialized to the particular situation and/or also allows multiple rules to be applicable in a given scenario is disclosed.

Conjunctive Rule Condition Table (CRCT). FIG. 5 is an illustration of an embodiment of a conjunctive rule condition table representation. In one embodiment, the representation of FIG. 5 may be used to represent rule condition tables (206) in FIG. 2A for the system of FIG. 1. As can be seen from its similarity, the rule condition table (502) of FIG. 5 may be considered a generalized version of the root cause table (222) of FIG. 2C.

Given a set of rules in which each rule condition is a conjunction of subconditions, it may be transformed into a table (502) with a row for each rule (504a), (504b), (504c), and so on, and a column, for example (506), for each subcondition occurring in a rule condition in the set of rules. The rowId or a label, for example (508), on the row directly or indirectly indicates the action to perform when the rule condition matches. The entry in row R and column C indicates the result required for the subcondition corresponding to column C to match this entry for the rule condition for the rule corresponding to row R. For example, if the subcondition for column C is (altimeter >200) and the entry in row R and column C is the value “true”, the altimeter reading at time T as an input may be greater than 200 Meters for this entry to match and thus the rule corresponding to row R to be activated.

An entry that corresponds to a subcondition that is not part of the corresponding rule condition stores a special “don't care” indication indicating that this entry matches for any input. This “don't care” designation is important because, in many applications, many rule conditions refer to a subset of subconditions that arise across all the rules. For example, in an HVAC system, a rule that depends on the compressor being turned off does not depend on the cooling coil temperature or pressure whereas when the compressor is on, these inputs are normally very relevant.

A rule condition matches when all the subconditions evaluate to the corresponding entry value or the entry value is “don't care”. Thus, a row corresponds to the conjunction of its subconditions. A table (502) in this format is referred to as a conjunctive rule condition table (CRCT). A CRCT is a representation of the rule conditions of a set of rules where there is a subcondition associated with each column (506) and each row (504a), (504b), corresponds to the conjunction of subconditions that corresponds to a rule condition of at least one rule in this set and that row is labelled (508) to identify, directly or indirectly, the action associated with this rule. As shown herein, Table A and Table B are conjunctions (&&) of subconditions such as the subcondition of a patient having a high body temperature.

In one embodiment, a column (506) may correspond to an expression that evaluates to more than two values. For example, a column corresponding to temperature range may evaluate to “cold”, “cool”, “normal”, “warm” and “hot”. In this case, each entry in the column may indicate one of these values or else “don't care”. Then, the entry matches if the temperature range value is equal to that stored in the entry.

In one embodiment, in which a column (506) may correspond to an expression that evaluates to multiple values, the entry may indicate a relational operator to apply as well as a value. For example, continuing the temperature example, the entry may indicate greater than “normal”. Thus, the column is labelled as “temperature relop value”, where relop is one of the relational operators and the value is the value stored in the entry. Other subcondition/entry representation schemes are feasible.

The CRCT representation (502) facilitates efficiently determining the subset of rules to use after the results of a test. This is an improvement for computing resources as it simplifies to removing each row that has a subcondition negated by the results of the test or conversely, including in the new subtable, those rows for which no subcondition is made false. Therefore, it is easier and more efficient to automatically produce the subset, or equivalently, the subtable after each test.

Converting Non-conjunctive Rule Conditions to Conjunctive. A ruleset may be converted to a conjunctive ruleset if it contains any disjunctions.

If a rule contains a disjunction in its condition, a new column corresponding to this disjunction may be introduced. For example, if there is a rule condition of the form:

if (SC0 and (SC1 or SC2)) then do action Foo

where SC0, SC1 and SC2 are subconditions, the new column logically labelled Sc1orSc2 may be introduced so the above rule becomes:

if (SC0 and SC1orSC2) then do action Foo

This conversion results in an additional subcondition column in the table. As shown herein, this demonstrates that any rule condition may be converted to a conjunctive form by adding more columns.

Another technique may be used in some cases to convert a rule condition to a simple conjunctive form without introducing new inputs, namely: if a rule contains a disjunction in its condition, the rule may be entered into the table as two rule conditions corresponding to the disjunction possibilities, as described in U.S. Patent Publication 2020/0081882, entitled AUTOMATIC GENERATION OF AN EFFICIENT RULE SET IMPLEMENTATION filed Sep. 5, 2019 which is incorporated herein by reference for all purposes. Using this technique, the above initial rule is thus transformed into two rows corresponding to:

if (SC0 and SC1) then do action Foo

if (SC0 and SC2) then do action Foo

Additional techniques from Boolean algebra may be used to transform rule conditions into a conjunctive form in some cases.

The additional columns to deal with disjunctions may incur extra storage and pre-computation cost. On the other hand, removing disjunction by duplicating a rule across multiple rows may increase the cost of matching. Using these techniques, one of ordinary skill in the art is able to transform any ruleset into a CRCT.

A table representation is an improved way to represent a ruleset, in part because it provides a more space-efficient and/or time-efficient representation than representing each rule separately. For instance, the designation of a column (506) associated with a particular subcondition may be done once and then be implicit for each entry in the column, rather than designating it for each instance of a rule condition in which it occurs. Also, some attribute selection methods entail processing across all the entries in a given column (506) for the ruleset.

This processing is more efficient when these subconditions are organized into columns (506). The conjunctive structure simplifies the structure and interpretation of a row (504) because the result is the same independent of the grouping of subconditions. By contrast, if a disjunction is allowed, the evaluation is sensitive to the grouping with respect to other conjunctions. For example (SC0 or (SC1 and SC2)) is different from ((SC0 or SC1) and SC2).

As described in U.S. patent application Ser. No. 17/156,378 (Attorney Docket OPTUP018) entitled AUTOMATIC GENERATION OF CONTROL DECISION LOGIC FOR COMPLEX ENGINEERED SYSTEMS FROM DYNAMIC PHYSICAL MODEL filed Jan. 22, 2021 which is incorporated herein by reference for all purposes, automatic generation of a CRCT (with associated actions) for a control system may be used. In one embodiment, the CRCT is represented as shown in FIG. 2C and in U.S. Pat. No. 10,761,921 with “don't care” subconditions.

In one embodiment, approximate matching as disclosed in U.S. Patent Publication No. 2020/0257692, entitled APPROXIMATE MATCHING filed Jan. 22, 2020 which is incorporated herein by reference for all purposes is used to identify a subtable of the original CRCT. In particular, if there are K known inputs after performing the last test, approximate matching is performed on the current CRCT with these K known inputs and a minimum match specified as K, with the resulting matched rows returned as a table. Because there are K known inputs and the minimum number of matches is required to be K, then the rows/rules that are not negated by the current known inputs are returned.

In one embodiment, the match refinement as in U.S. Patent Publication No. 2019/0294486, entitled MATCHSET-BASED AUTOMATIC ROOT CAUSE ANALYSIS filed Mar. 19, 2019 which is incorporated herein by reference for all purposes, is used to suppress some matched potential root cause failures and order root cause failures by probability. Probabilities may thus be associated with outputs.

System Root Cause Fault Identification. The disclosed is applicable to root cause fault identification in complex systems where not all the telemetry/symptoms are feasible to measure at all times. For example, a specific problem in an aircraft may indicate an engine problem but determining the exact nature of the engine problem may require taking the engine out of service and attaching the engine to a diagnostic system for further diagnosis. In other cases, it may be necessary to physically open up some component to test its internal operating parameters because these parameters are not exposed externally. In both cases, significant costs are involved to perform these detailed tests so they are performed after at least one of the problems has been identified to associated with that component.

An alternative approach is to use a first stage of rules to identify the general problem or fault as associated with a particular component and then a second stage RCI ruleset specific to this component to root cause the problem within the component. However, this alternative interferes with the model-based generation of rules for RCI as described in U.S. Pat. No. 10,761,921. In particular, U.S. Pat. No. 10,761,921 and U.S. Patent Publication 2020/0081882, and also described in U.S. Patent Publication 2021/0240148, entitled AUTOMATIC GENERATION OF CONTROL DECISION LOGIC FOR COMPLEX ENGINEERED SYSTEMS FROM DYNAMIC PHYSICAL MODEL filed Jan. 22, 2021 which is incorporated herein by reference for all purposes, teach the generation of rules from a model, either a fault model for RCI or an operational model for control.

Having a more detailed model provides a more accurate set of rules, and thus better end-application behavior. In many cases, the interactions of one component with others to which it is connected are critical to determine the actual root cause fault. Therefore, providing a high-level model to classify into broad categories is not feasible because the accurate classification is dependent on many specific low-level details. For example, in an HVAC system, the exact problem with the compressor may be indicated by the behavior of other components such as cooling coil and current sensor. Therefore, a hierarchical classification is not always a viable alternative.

This same problem may arise in other domains. For instance, in the medical diagnosis domain, a disorder may induce symptoms across a range of organs and systems in the body and also general body symptoms. It may not be feasible to root cause a disorder to a particular organ or system with high-level symptoms. Moreover, even when a hierarchical classification is feasible technically, the sequence of tests required to achieve this classification may lead to a more expensive test sequence.

In any event, the hierarchy classification may use the incremental rule evaluation at each level in the hierarchy to reduce the cost of achieve a decision on the classification.

Hypothesis Evaluation. In some applications, the operator may have an initial hypothesis of the problem. In one embodiment, an initial hypothesis may be entered into the system. The system then selects the initial subset of rules that correspond to this hypothesis by selecting the rules whose actions correspond to this hypothesis. In this embodiment, the system selects the input attribute that is most effective in confirming or contradicting the hypothesis by applying the disclosed to this subset.

For example, if the hypothesis is leukemia, there may be a subset of rules whose action corresponds to a diagnosis of leukemia. Using that rule subset, the attribute selection then selects the input to determine based on the one that reduces this subset the most. In a preferred embodiment, this selected input may result in confirming or disproving the hypothesis; that is, if the subset is reduced to the empty set, the hypothesis is contradicted because there is no rule condition in the subset corresponding to this hypothesis that may be true. If there is exactly one rule that corresponds to this hypothesis, the disclosed may determine effectively whether that given rule condition is true.

In one embodiment, the current subset may be reduced by filtering on the actions associated with the rules, removing those rules whose actions do not pass the filter. The filtering may be performed on the initial rule set. It may also be separately performed on iterations of reduction based on input value determination. The initial reduction corresponds to an initial hypothesis. Subsequent reductions based on actions correspond to a revised hypothesis or hypotheses.

Reducing the need for hypothesis-based diagnosis is disclosed in part as an automated system may record and evaluate a large number of hypotheses in parallel with the efficiencies of the CRCT. This approach is an improvement as it avoids the trial-and-error inefficiency of hypothesis-based diagnosis. It also is consistent in evaluating less probable diagnosis rather than just focusing on common diagnoses.

Medical Diagnosis Application. Medical diagnosis is a domain in which all potentially useful “telemetry” about a patient is not known at the point of evaluation, for example when the patient is freshly admitted to a hospital. Moreover, some diagnostic tests are quite expensive to perform and may be a risk or inconvenience to the patient. Therefore, medical diagnosis applies a cost-effectiveness analysis (CEA) model and incremental cost-effectiveness ratio (ICER) to determine the course of testing, diagnosis and treatment. The medical field has a notion of diagnostic yield defined as the likelihood that a test or procedure provides the information needed to establish a diagnosis.

In the medical domain, there may be different test results/history available for each patient. There may also be different rulesets for different categories of patients such as “young”, “old”, “teenage”, “male”, and/or “female, as well as based on the apparent seriousness of the condition.

In the medical domain, it may be necessary to start an intervention, such as having the patient take a particular medicine, before a fully confident diagnosis is available. This intervention may not immediately produce results and is also dependent on the hypothesized diagnosis being correct; that is the test is prescribing an intervention that should produce a significant improvement if the diagnosis is correct. Note that a treatment and the measured response to the treatment may also be considered a test.

In the medical domain, detecting an adverse reaction or sudden degradation in condition in a patient calls for immediate remedial/precautionary action even before there is a full understanding of the cause of the adverse reaction.

All of these factors of the medical domain make traditional RCI inapplicable because it requires all the symptoms available at the point of evaluation. Conventional medical diagnosis often relies on the physician forming a hypothesis of the disorder the patient has, and then selecting tests and treatment based on this hypothesis.

An automated diagnostic system as disclosed herein, is an improvement over the traditional physician as it is feasible to keep a massive number of potential diagnoses, corresponding to those remaining in the rule subset, under consideration concurrently, far more than a human may be expected to recall and manage.

Another improvement of the incremental evaluation of a ruleset is avoiding bias that is introduced by the hypothesis-based approach. In particular, it reduces the frequency of misdiagnosis where a common ailment and/or one a physician has experience with is diagnosed based on the hypothesis when the actual disorder is an uncommon one and/or one a physician has less experience with has similar symptoms. This is because even rare ailments are kept under consideration in the rule subset until each has been eliminated by the results of tests.

Another improvement of the incremental evaluation of a ruleset is that it may also accommodate hypothesis-based diagnosis, as described above. In some cases, medical diagnosis is dependent on X-rays and CAT scans that effectively require image processing. In these cases, the incremental evaluation of a ruleset may be used in conjunction with an interpretation of the image, such as that provided by a radiologist. In these cases, the image processing may translate the images to symptoms, and the incremental evaluation of a ruleset is then able to factor this information together with other inputs to perform the root cause analysis or diagnosis.

More generally, images may be part of the selected input and/or used to perform a diagnosis. For example, a patient's temperature, blood pressure, and/or heart rate may also be measured. In this case, the image processing output may be treated as essentially one input to the rule-based diagnosis as described herein. Still, the candidate image interpretations may nevertheless be refined by using incremental evaluation of a ruleset against the image interpretation, possibly with this additional telemetry as input. For example, high heart rate may be a counter-indicator to some interpretation of an image of the heart.

A medical treatment plan may be viewed as a “lane” for navigating a patient back to health, potentially corresponding to a sequence of treatment. Thus, the “lane-structured” environment patent U.S. Patent Publication No. 2020/0264900, entitled USING A LANE-STRUCTURED DYNAMIC ENVIRONMENT FOR RULE-BASED AUTOMATED CONTROL filed Feb. 19, 2020 which is incorporated herein by reference for all purposes applies to medical treatment. That is, the number of sequences of treatment is constrained to those established sequences in treatment that are known to safe based on prior clinical studies. For example, a patient should not be prescribed to take medicine X if already taking medicine Y. This constraint limits the potential treatment plans, making the number of actions a practical number to handle within a ruleset.

Investment Decision Making Application. In investment and business decisions, a variety of options are often available. To make a business or investment decision on these options, there is often a ruleset to be evaluated against an option to determine if it is a compelling opportunity. For example, to provide a simple case, one rule might be: if cost basis is highly dependent on economics of scale, determine total addressable market and expected rate of market penetration. It is often necessary to investigate issues at the cost of significant time and effort.

Therefore, incrementally evaluating the rule set based on incrementally determining the inputs, such as total addressable market and expected rate of market penetration allows an investment opportunity to be assessed at lower cost and potentially faster, especially if it turns out to be unattractive. That is, it may be eliminated by one test before evaluating all the other inputs required to fully evaluate all the rules as applied to this opportunity. Consequently, incremental evaluation of a ruleset may be performed as described herein when a ruleset is used in decision making with business or investments.

Other Improvements over Traditional Techniques. Incremental evaluation of a ruleset differs from traditional methods for rule-based generation of decision trees because no decision tree need be generated, saving storage, network, and/or computing resources. By contrast, incremental evaluation of a ruleset generates the next decision node/test dynamically, based on previous results and a cost-information gain function that is specific to the subject system such as a patient being diagnosed. This sequence is equivalent to generating the decision path dynamically except that each next interior node is dependent on the results of the previous test. For example, if a blood test reveals low red cell count, the subset of the rules that has that value or “don't care” is considered in selecting the next node. This contrasts with the computing inefficiencies and static nature of instantiating a decision tree and then using it repeatedly for decisions.

Another improvement of incremental evaluation of a ruleset is that it does not change the values used in an earlier evaluation of the rules. Incremental evaluation of a ruleset is not the same as incremental “re-evaluation” without providing any guidance on the order of test or input evaluation beyond reacting to revised inputs.

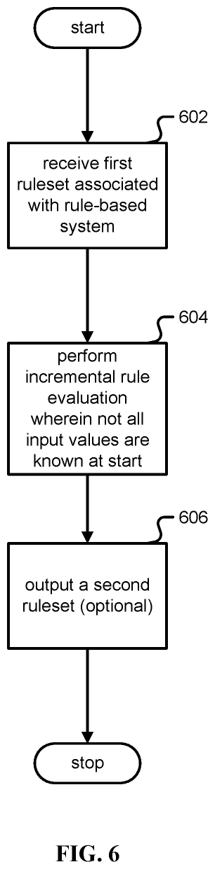

FIG. 6 is a flow diagram illustrating an embodiment of a process for incremental evaluation of a ruleset. In one embodiment, the process of FIG. 6 is carried out by the system of FIG. 1.

In step (602), a first ruleset associated with a rule-based system is received. In one embodiment, the first ruleset is reduced prior to performing incremental rule evaluation, at least in part by receiving an already known input value and removing from the first ruleset rules whose rule condition can no longer be true based on the already known input value. In one embodiment, the first ruleset is reduced prior to performing incremental rule evaluation, at least in part by receiving a hypothesis and removing from the first ruleset rules whose actions do not correspond to the hypothesis.

In one embodiment, the first ruleset is applied to root cause identification. In one embodiment, the first ruleset is applied to medical diagnosis. In one embodiment, the first ruleset is applied to investment decision making.

In step (604), incremental rule evaluation is performed, wherein not all input values are known at the start of rule evaluation. In one embodiment, memory usage associated with the first ruleset is freed after performing the incremental rule evaluation of step (604). In one embodiment, performing incremental rule evaluation further comprises reducing the first ruleset at least in part by filtering out rules based on their associated actions.

In one embodiment, the first ruleset is represented by a conjunctive rule condition table (CRCT), wherein: each rule condition in the first ruleset is represented a conjunction of subconditions; each rule is represented by a row in the CRCT; and each subcondition occurring in a rule condition in the first ruleset is represented by a column in the CRCT. In one embodiment, an entry in the CRCT may be a “don't care” value indicating that the entry matches for any input.

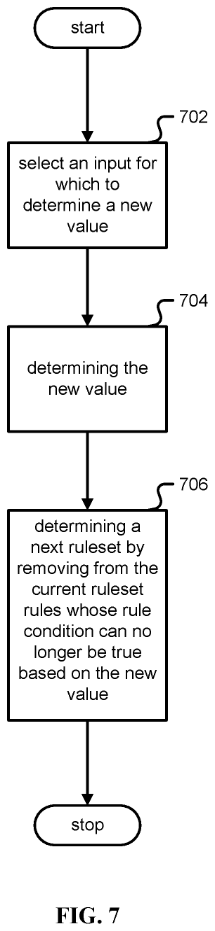

FIG. 7 is a flow diagram illustrating an embodiment of a process for performing incremental rule evaluation. In one embodiment, the process of FIG. 7 is at least part of step (604) in FIG. 6.

In step (702), a first input for which to determine a first new value is selected. In one embodiment, selecting the first input is based at least in part on expected information gained from determining the first input value. In one embodiment, selecting the first input is based at least in part on the cost of determining the first input value. In one embodiment, selecting the first input is based at least in part on an attribute selection process. In one embodiment, selecting the first input is based at least in part on an RBDT-1 process. In one embodiment, selecting the first input is sensitive to a scenario in which rules are being evaluated.

In step (704), the first new value is determined. In step (706), a second ruleset is determined at least in part by removing from the first ruleset rules whose rule condition can no longer be true based on the first input value.

In the event the first ruleset is not sufficiently reduced when the second ruleset is determined, further incremental rule evaluation is performed, comprising a second iteration similar to that shown of FIG. 7, wherein: a second input is selected for which to determine a second new value; the second new value is determined; and a third ruleset is determined by removing from the second ruleset rules whose rule condition can no longer be true based on the second input value. Without limitation, this incremental rule evaluation iteration of FIG. 7 may be repeated when the current ruleset is not sufficiently reduced.

Returning to FIG. 6, in optional step (606), the second ruleset is output. In one embodiment, this includes performing actions associated with the second ruleset. In one embodiment, this includes receiving one or more additional inputs, and further evaluating the rules, for example applying actions of rules associated with the second ruleset whose condition cannot be determined to evaluate as false based at least in part on the additional input.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A method, comprising:
receiving a first ruleset associated with a rule-based system; and
performing incremental rule evaluation wherein not all input values are known at the start of rule evaluation, at least in part by:
selecting a first input for which to determine a first new value;
determining the first new value; and
determining a second ruleset by removing from the first ruleset rules whose rule condition can no longer be true based on the first input value.

2. The method of claim 1, wherein performing incremental rule evaluation further comprises, in the event the first ruleset is not sufficiently reduced:
selecting a second input for which to determine a second new value;
determining the second new value; and
determining a third ruleset by removing from the second ruleset rules whose rule condition can no longer be true based on the second input value.

3. The method of claim 1, further comprising reporting the second ruleset.

4. The method of claim 1, further comprising:
outputting the second ruleset, at least in part by performing actions associated with the second ruleset.

5. The method of claim 1, further comprising:
outputting the second ruleset, at least in part by:
receiving an additional input; and
applying actions of rules associated with the second ruleset whose condition cannot be determined to evaluate as false based at least in part on the additional input.

6. The method of claim 1, further comprising freeing memory usage associated with the first ruleset after performing incremental rule evaluation.

7. The method of claim 1, wherein selecting the first input is based at least in part on expected information gained from determining the first input value.

8. The method of claim 1, wherein selecting the first input is based at least in part on the cost of determining the first input value.

9. The method of claim 1, wherein selecting the first input is based at least in part on an attribute selection process.

10. The method of claim 1, wherein selecting the first input is based at least in part on an RBDT-1 process.

11. The method of claim 1, wherein selecting the first input is sensitive to a scenario in which rules are being evaluated.

12. The method of claim 1, wherein performing incremental rule evaluation further comprises reducing the first ruleset at least in part by filtering out rules based on their associated actions.

13. The method of claim 1, further comprising reducing the first ruleset prior to performing incremental rule evaluation, at least in part by receiving an already known input value and removing from the first ruleset rules whose rule condition can no longer be true based on the already known input value.

14. The method of claim 1, further comprising reducing the first ruleset prior to performing incremental rule evaluation, at least in part by receiving a hypothesis and removing from the first ruleset rules whose actions do not correspond to the hypothesis.

15. The method of claim 1, wherein the first ruleset is represented by a conjunctive rule condition table (CRCT), wherein:
each rule condition in the first ruleset is represented a conjunction of subconditions;
each rule is represented by a row in the CRCT; and
each subcondition occurring in a rule condition in the first ruleset is represented by a column in the CRCT.

16. The method of claim 15, wherein an entry in the CRCT may be a “don't care” value indicating that the entry matches for any input.

17. The method of claim 1, wherein the first ruleset is applied to root cause identification.

18. The method of claim 1, wherein the first ruleset is applied to medical diagnosis.

19. The method of claim 1, wherein the first ruleset is applied to investment decision making.

20. The method of claim 1, wherein determining the first new value is performed at evaluation time.

21. A system, comprising:
a communication interface;
a processor coupled to the communication interface and configured to:
receive a first ruleset associated with a rule-based system from the communication interface; and
perform incremental rule evaluation wherein not all input values are known at the start of rule evaluation, at least in part by:
select a first input for which to determine a first new value;
determine the first new value; and
determine a second ruleset by removing from the first ruleset rules whose rule condition can no longer be true based on the first input value.

22. A computer program product embodied in a non-transitory computer readable medium and comprising computer instructions for:
receiving a first ruleset associated with a rule-based system; and
performing incremental rule evaluation wherein not all input values are known at the start of rule evaluation, at least in part by:
selecting a first input for which to determine a first new value;
determining the first new value; and
determining a second ruleset by removing from the first ruleset rules whose rule condition can no longer be true based on the first input value.
